(19) United States
(12) Patent Application Publication
(10) Pub. No.: US 2022/0145369 A1
(43) Pub. Date: May 12, 2022

(54) A NOVEL SAMPLING METHOD FOR LONG-TERM MONITORING OF MICROBES

(71) Applicant: MERCK PATENT GMBH, DARMSTADT (DE)

(72) Inventors: Martin BOBAL, Giesshuebl (AT); Anna WITTE, Wien (AT); Patrick MESTER, Wien (AT); Peter ROSSMANITH, Gaaden (AT)

(73) Assignee: MERCK PATENT GMBH, DARMSTADT (DE)

(21) Appl. No.: 17/438,902

(22) PCT Filed: Mar. 12, 2020
(86) PCT No.: PCT/EP2020/056581

§ 371 Date: Sep. 13, 2021

(30) Foreign Application Priority Data
Mar. 14, 2019 (EP) 19162946.8

(51) Int. Cl. C12Q 1/689; C12Q 1/6806

(52) U.S. Cl. CPC C12Q 1/689; C12Q 1/6806

(57) ABSTRACT

The invention relates generally to the field of detection of biological contaminants on a surface, specifically to a method comprising the steps of providing one or more pieces of sterile and nucleotide-free adhesive fibrous material, affixing said one or more pieces of the fibrous material to said surface, collecting said one or more pieces of the fibrous material from said surface, incubating said one or more pieces of the fibrous material in a solvent, and analyzing the solvent for the presence of biological contaminants. The invention further relates to a kit of parts comprising sterile carriers and instructions to be used for long-term monitoring of microbes.

### FIELD OF THE INVENTION

The invention relates generally to the field of detection of biological contaminants on a surface, specifically to a method comprising the steps of providing one or more pieces of sterile and nucleotide-free adhesive fibrous material, affixing said one or more pieces of the fibrous material to said surface, collecting said one or more pieces of the fibrous material from said surface, incubating said one or more pieces of the fibrous material in a solvent, and analyzing the solvent for the presence of biological contaminants. The invention further relates to the sterile carriers and to a kit of parts comprising sterile carriers and further parts like instructions to be used for long term monitoring of microbes.

### BACKGROUND OF THE INVENTION

Foodborne pathogens can cause serious diseases and death. Further, recalls of potentially contaminated goods result in significant economic damage. A prominent example is Listeria monocytogenes, a Gram-positive, facultative anaerobic and ubiquitous human pathogen, which has the ability to adhere to surfaces commonly encountered in food processing environments (Silva S, et al., J Food Prot. 2008; 71(7):1379-1385). Outbreaks of listeriosis continue to occur sporadically and the mortality rate is as high as 20%. Since L. monocytogenes is able to grow under refrigeration conditions (4° C., Gandhi M, et al., Int J Food Microbiol. 2007; 113(1):1-15) and as clinical symptoms are frequently compiled only after delays, listeriosis is an especially serious diagnosis. For these reasons national and international standards and regulations for cleansing, disinfection and monitoring foodborne pathogens have implemented zero tolerance for L. monocytogenes in ready-to-eat foods (Public Health England. Detection and Enumeration of Bacteria in Swabs and Other Environmental Samples.; 2017; Pueyo a E, et al., Guidelines on sampling the food processing area and equipment for the detection of Listeria monocytogenes. 2012:15 www.anses.fr.). However, only the most reliable methods with lowest detection limits will achieve this goal. Common assays for monitoring pathogens comprise conventional microbiological methods based on growth and are thus time consuming to perform (Anonymous; Microbiology of Food and Animal Feeding Stuffs—Horizontal Methods for Sampling Techniques from Surfaces Using Contact Plates and Swabs. International Organization for Standardization: Geneva, Switzerland; 2004). In order to reduce processing time and costs, faster and more reliable alternative detection methods are being investigated.

A promising departure from growth-based methods is the quantitative polymerase chain reaction (qPCR), which provides faster results. Theoretically, the detection limit of this method approaches one copy of the target gene (Rossmanith P, and Wagner M., J Food Prot. 2011; 74(9):1404-1412). Practically, it is an especially sensitive tool that has undergone enormous developmental progress over the past several years. Although qPCR cannot distinguish between living and dead cells, results reflect the occurrence of (present or past) contamination, including the presence of viable but non-culturable cells (VBNC).

However, a prerequisite for accurate microbiological data is effective sampling. In the food production facility, conventional swabbing as a standard method can only expose a momentary snapshot. For example, it is not possible to reconstruct information about yesterday's status after cleansing has been performed. In addition, when moistened swabs or contact-plate sampling methods are used, they bring with them growth medium into a supposedly clean environment making subsequent disinfection necessary. To obtain comparable results, swabbing should be performed within a defined area with reference to ISO 18593 (Anonymous, 2004) or the FSIS directive. This is not very practical with complex surfaces such as door handles, light switches and other typical fomites. The method itself intrinsically has a low ability to take up bacteria from dry surfaces, and is associated with highly variable recovery rates averaging 20 (Witte A K, et al., LWT—Food Sci Technol. 2018; 90, 186-192).

Therefore, there is a clear need in the field for an improved sampling method which allows effective and thorough sampling, in particular for long-term monitoring, and preferably is still cost-efficient.

### SUMMARY OF THE INVENTION

Detection of pathogens is crucial in production areas. Such contaminants may be found on equipment or other surfaces used in environments including food processing plants, pharmaceutical production facilities, hospitals, veterinary offices, and restaurants. The need for feedback to cleaning and audit personnel on the presence of residual contaminating substances in a variety of environments is well-established. For example, the need for contaminant monitoring has a well-documented role in food safety programs when residual food residues can result in bacterial contamination and allergic responses in some individuals. Effective cleaning also reduces the risk of pathogens contaminating subsequent food products. A variety of devices and methods have been utilized for contaminant testing. Similarly, there is a need to ensure that surfaces and equipment in hospitals, physicians' offices, clinical laboratories, or veterinarian offices have been adequately cleaned to protect patients and staff. While being well established, swabbing as a state-of-the-art sampling method offers several drawbacks in respect of yield, standardization, overall handling and long-term monitoring.

It is the objective of the present invention to provide an improved method for detecting microbes employing a method which is highly sensitive, easy to use, rapid and inexpensive.

The objective is solved by the present invention.

According to the invention, there is provided a method for the detection of biological contaminants on a surface, comprising the sequential steps of

i. providing a carrier comprising one or more pieces of sterile fibrous material and an adhesive part, preferably an adhesive layer, line or dots,
ii. affixing said carrier to said surface,
iii. leaving the carrier affixed to said surface for a suitable time
iv. collecting said carrier from said surface,
v. incubating at least the fibrous material of the carrier in a solvent, and
vi. analyzing the solvent for the presence of biological contaminants.

Specifically, at least 2 pieces of fibrous material are used, specifically at least 3, 4, 5 or 6 pieces of fibrous material are used.

According to a specific embodiment, the fibrous material is comprised on an adhesive support capable of adhering to the surface.

Specifically, the surface is a non-biological surface.

Specifically, the fibrous material is comprised on a layer of paper or layer of another material like a plastic layer which is adhered to an adhesive support capable of adhering to the surface.

According to an embodiment of the invention, the adhesive carrier comprises at least two sections, optionally separated by a perforated line, wherein at least one section comprises one or more pieces of sterile and preferably nucleotide-free adhesive fibrous material and wherein at least one section does not comprise the fibrous material.

According to a further embodiment of the invention, the adhesive carrier comprises at least two sections separated by a perforated line and the one or more pieces of fibrous material are situated on the perforated line, and wherein the adhesive carrier optionally comprises at least one non-adhesive section.

According to an embodiment, the biological contaminants which can be detected by the method as described herein are bacteria, specifically Listeria monocytogenes or E. coli, fungi, like e.g. yeast, or viruses and any combinations thereof.

According to an embodiment, the solvent used for the method described herein is specifically selected from the group consisting of buffers, specifically selected from the group of buffers with solvents, surfactants, detergents, buffers without solvents, surfactants, detergents; Tris/EDTA; chaotropic solvents, organic solvents, ionic liquids.

According to an embodiment of the invention, the solvent is analyzed for parts of a biological contaminant selected from the group consisting of proteins, peptides, and nucleic acid molecules, specifically DNA or RNA.

According to a specific embodiment, analysis for the biological contaminant or parts of a biological contaminant is employed by using PCR, qPCR, next generation sequencing (NGS), enzyme-linked immunosorbent assay (ELISA) or any other immunoassays.

Specifically, the biological contaminant is L. monocytogenes and the solvent is analyzed for the presence of the L. monocytogenes gene prfA.

According to an alternative embodiment, the biological contaminant is E. coli and the solvent is analyzed for the presence of the E. coli gene sfmD.

Specifically, according to the method described herein, the fibrous material is affixed to the surface for at least 1 hour, 6 hours, 8 hours or 12 hours, preferably at least 24 hours.

In an alternative embodiment, the fibrous material is affixed to the surface for at least a week, preferably at least 2 weeks. Overall the time for which the carrier stays affixed to the surface depends on the type of monitoring that shall be performed. Short term monitoring might only cover one production shift or the time between two cleanings, e.g. 1 to 12 hours. Mid-term monitoring might cover 4 to 48 hours and long time monitoring might cover 48 hours up to 2 to 4 weeks.

According to a specific embodiment, the carrier is sterilized using a physical or chemical sterilization method, specifically selected from the group consisting of UV radiation, gamma radiation, electron beam radiation, X-ray radiation, radiation with subatomic particles, plasma, dry heat, autoclaving, ozone, hydrogen peroxide, peracetic acid, nitrogen dioxide, ethylene oxide, hypochlorite and DNase.

Specifically, the fibrous material is inorganic or organic fibrous material, specifically selected from the group consisting of activated carbon, microporous ceramic, porous metal, aluminium oxide, glass fiber, paper, cellulose, cellulose esters, cellulose ethers, cellulose acetate, viscose, cellophane, alginate, nylon membranes, polyester (PETE), polypropylene, polytetrafluoroethylene (PTFE), polyvinylidene fluoride, polyvinylidene difluoride (PVDF), polycarbonate (PCTE), polyether ether ketone (PEEK), polyacrylonitrile (PAN), polyaramide (KEVLAR), and polyethersulfone (PES).

Specifically, the adhesive carrier is selected from the group consisting of adhesive tapes, specifically selected from the group consisting of polyethylene film, polypropylene film, polyester film, polyvinyl chloride (PVC), cellulose film, plastic paraffin film, and metal foil.

In a specific embodiment, the one or more pieces of fibrous material comprise a surface area of at least 10 mm², preferably of about 50 to 300 mm², more preferably of about 50 to 100 mm².

Specifically, the one or more pieces of fibrous material, the optional layer of paper or another material and/or the adhesive support are supplemented with a bacteriocide or bacteriostatic composition.

In a specific embodiment of the invention, the method is used for long-term monitoring of biological contaminants, specifically long-term monitoring, of biological contaminants in the food industry or in the medical or pharmaceutical sector.

Further provided herein is a kit of parts comprising at least the following parts:
i. at least one sticker comprising a sterile carrier comprising a first and second surface, wherein said first surface is adhesive and said second surface comprises at least one piece of sterile and nucleotide-free adhesive fibrous material and wherein said sticker is covered by a top and bottom sterile protective layer, and
ii. an instruction leaflet including a protocol for the detection of biological contaminants according to the method described herein.

### FIGURES

FIG. 1: Quantification of L. monocytogenes and E. coli from artificially contaminated stickers over a broad dynamic range. DNA from stickers artificially contaminated with four 10-fold logarithmic dilutions (starting at 80 cfu for E. coli and 10 cfu for L. monocytogenes) was extracted and quantified using qPCR (y axis). Control DNA (input, applied on stickers) was extracted and analyzed simultaneously as reference (x axis). Symbols and error bars denote standardized mean differences and standard deviation, respectively (n=3 independent experiments with three repetitions each). For the sake of clarity only positive y-error bars of are displayed (negative y-error bar values are identical to the positive values).

FIG. 2. Schematic representation of the artificially contaminated sticker setup. UVC-treated stickers were artificially contaminated by the addition of diluted bacteria suspensions at desired concentrations. After respective incubation times, DNA from stickers and controls was extracted and analyzed using qPCR. In parallel, cells were plated on TSA plates as controls.

FIG. 3. Recovery after cleansing and disinfection. Surfaces or stickers applied to surfaces were artificially contaminated with 10³ to 10⁴ cfu of L. monocytogenes ΔprfA. After drying, surfaces were washed, subsequently sampled and DNA extracted and analyzed with qPCR. Bars represent the grand mean of recovery (outcome (qPCR)/input (qPCR)) with the standard error of five independent experiments performed in duplicate.

FIG. 4. Accumulation of synthetic IAC on stickers over time qPCR (IAC assay) of DNA extracted from stickers applied to a door handle demonstrates an accumulation over time of synthetic DNA on stickers that was distributed in this room. Results are representative of three independent experiments.

FIG. 5. Stability of recovery over time. Stickers were artificially contaminated with L. monocytogenes ΔprfA and DNA extracted and analyzed with qPCR after 0, 1, 3, 7 and 14 days. Bars and errors bars represent grand means of recovery (outcome (qPCR)/input (qPCR)) and standard errors of four (days 0, 3) or three (1, 7, 14 days) independent experiments, including two different bacterial concentrations in duplicate.

FIG. 6. Pooling of stickers. a. Schematic representation of the pooling approach demonstrates the different samples: one single contaminated sticker, a single sticker with 1/6 contamination level, a pool containing six contaminated stickers and a pool containing one contaminated and five empty stickers. b. Results show that pooling of six stickers does not lead to a great loss of information. BCE (bacterial cell equivalents) were determined using qPCR. Bars represent the standardized mean difference with the standard deviation of four independent experiments.

FIG. 7. Schematic representation of the kit of parts:

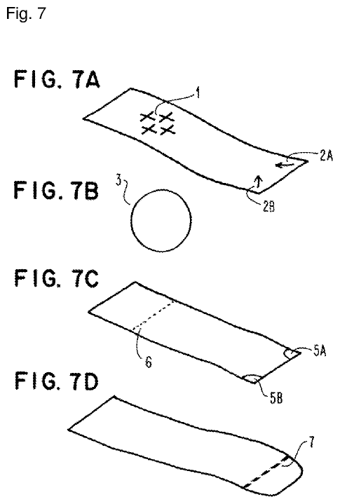

Upper protective layer (FIG. 7A) with position indicator (1) and non-adhesive section (2A)(2B); self-adhesive fibrous material (3) (FIG. 7B); adhesive carrier (FIG. 7C) with non-adhesive section (5A)(5B) and perforated line (6); lower protective layer (FIG. 7D) with layer separation guide (7).

### DETAILED DESCRIPTION

Unless indicated or defined otherwise, all terms used herein have their usual meaning in the art, which will be clear to the skilled person. Reference is for example made to standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Janeway et al, “Immunobiology” (5th Ed., or more recent editions, Garland Science, New York, 2001).

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

The method described herein refers to the detection of biological contaminants, including but not limited to food processing plants, pharmaceutical manufacturing facilities, hospitals, medical offices, veterinary offices, and restaurants.

Biological contaminants as referred herein can be any living organism or product that can harm animals and humans and compromise food safety and suitability, including microorganisms such as bacteria, viruses, fungi like yeasts and molds, and parasites.

Examples of biological contaminants can be, but are not limited to Giardia, such as G. lamblia. G. duodenalis, and G. intestinalis; Cryptosporidium, such as C. parvum, C. Jells, C. muris, C. meleagridis, C. suis, C. canis, and C. hominis; Salmonella, Shigella, Campylobacter, Corynebacterium, Candida, E. coli, Yersinia, Aeromonas, Microsporidia or other small pathogenic organisms. Specifically in food industry, Salmonella, Staphylococcus and Listeria are highly relevant contaminants. Biological contaminants can also be foodborne viruses, such as, but not limited to hepatitis A virus, hepatitis E virus, norovirus, human rotavirus, Nipah virus, highly pathogenic avian influenza virus, SARS-causing coronavirus, Campylobacter spp., and Streptococcus. According to an aspect of the present disclosure, the presence of one or more species may be captured and detected with the method described herein. In particular, the method is well suited to detecting one or two pathogens, but more or different types of organisms may also be targeted and analyzed.

As specified herein, a sterile and preferably nucleotide-free carrier comprising an adhesive part and a fibrous material is used for the detection method.

The term “fibrous material” refers to an inorganic or organic fibrous material comprising suitable pores to immobilize, entrap, capture or adhere biological contaminants which can be released upon contacting the material with a suitable solvent. The material can be, but is not limited to, inorganic material such as activated carbon, charcoal, microporous ceramic, porous metal, aluminium oxide, glass fiber, or organic material such as paper, cellulose, cellulose esters, cellulose ethers, cellulose acetate, viscose, cellophane, alginate, nylon membranes, polyester (PETE), polypropylene, polytetrafluoroethylene (PTFE), polyvinylidene fluoride, polyvinylidene difluoride (PVDF), polycarbonate (PCTE), polyether ether ketone (PEEK), polyacrylonitrile (PAN), polyaramide (KEVLAR®), and polyethersulfone (PES). In a specific embodiment, the fibrous material is paper.

Preferably, the material used should be bacteriostatic and widely insensitive to moisture or abrasion. It may also incorporate adhesive mechanisms for cell membranes. It should neither inhibit DNA-extraction nor the performance of qPCR. Therefore, it preferably does not contain any nucleic acids which could hinder or influence the analysis of the contaminants, or it does not contain any nucleic acids at all.

The fibrous material can be sterilized and nucleic acids can be removed by any method known in the art and adjusted to the respective fibrous material. Such methods can be, but are not limited to, physical sterilization such as radiation (UV-C, gamma, electron beam, X-ray, subatomic particles), plasma (ionized gas), dry heat, autoclaving (steam); chemical sterilization methods such as treatment of the fibrous material with ozone, hydrogen peroxide, peracetic acid, nitrogen dioxide, ethylene oxide, hypochlorite, and DNase.

The fibrous material can be affixed to any surface assumed to be contaminated or to be at risk to contamination. The term “affixing” refers to stick, attach, or fasten the fibrous material to a surface in a way that the material can be removed from said surface for further analysis. Typically, the fibrous material is affixed to a surface so that the fibrous material faces into the interior of the room, away from the surface. The pieces of the fibrous material can be affixed to the surface for any period of time considered appropriate for detection of biological contaminant. The period may last for 1, 2, 3, 4, 5, 6, 7, 8, 9, 10, 11, 12 or more hours, preferably at least 24 hours. The period wherein the fibrous material is affixed to the surface may also be longer, such as 1, 2, 3, 4, 5, 6, or more weeks. Due to the fibrous composition of the material, biological contaminants are fixed and at least residues or traces of the contaminants are preserved for analysis even if the surface is repeatedly cleaned.

As used herein the term “non-biological surface” refers to a non-living surface such as that of an inert object or structure.

In a specific embodiment, the fibrous carrier may be supplemented with a bacteriocide or bacteriostatic composition. A bacteriostatic agent or bacteriostat, is a biological or chemical agent that stops bacteria from reproducing, while not necessarily killing them otherwise. Depending on their application, bacteriostatic antibiotics, disinfectants, antiseptics and preservatives can be distinguished. A bactericide or bacteriocide is a substance that kills bacteria. Bactericides can be disinfectants, antiseptics, or antibiotics.

According to the invention, the fibrous material can be directly affixed to the adhesive part. Thereby the material comprises on its bottom side an adhesive material or support capable of adhering to the surface. Specifically, the fibrous material can be paper comprising as the adhesive part an adhesive glue on one side or on a part of one side which can be affixed and removed from a surface. Specifically, the pieces of paper can comprise a low-tack pressure-sensitive adhesive well known for Post-It® sticker. The adhesive part may also be a sheet or layer, e.g of paper, metal or plastic with an adhesive. The adhesive may cover one whole side of the layer or it may be applied to only a part of the layer, e.g. in form of a line or one or more dots.

An adhesive part according to the invention is also a part which is not per se adhesive in the sense of it being sticky but which can be generally attached to a surface. It may for example be a fixation means or a holding which can be otherwise fixed to a surface. In this case, the carrier for example comprises the fibrous material and an e.g. metal or plastic holding that can be permanently attached to a surface, e.g. with an adhesive or with other fixation means like nails, screws etc. After the period wherein the fibrous material shall be affixed to the surface, it can be removed from the holding and can be further analysed while the holding stays attached to the surface and a new fibrous material can be inserted any time. This embodiment ensures that the position of the fibrous material at the surface is permanently the same, even over several rounds of analysis. In addition, in one embodiment the fibrous material only needs to be removed from the holding which is fixed to the surface after the incubation period and can be further analysed without the adhesive part which might otherwise disturb the further analysis.

The size of the pieces of fibrous material is not essential and can be adapted to the size of the surface to be tested for contamination. The smaller the respective surface, the smaller the piece of fibrous material is defined. The fibrous material may have a surface area of at least about 5 mm², about 10 mm², specifically of about 50 to 300 mm², more specifically of about 50 to 100 mm².

The pieces of fibrous material can be of any shape, such as but not limited to rectangle, square, triangle, round or ellipsoid.

According to an alternative, the fibrous material can is fixed to an adhesive part. Said adhesive part can be any material, such as but not limited to adhesive tapes well known, pressure sensitive adhesive tapes, polyethylene films, polypropylene films, polyester films, polyvinyl chloride (PVC), cellulose films, plastic paraffin films, or metal foils.

According to an alternative method, the carrier comprises an adhesive part with two or more sections, optionally separated by a perforated line, wherein one or more sections comprises one or more pieces of sterile and nucleotide-free adhesive fibrous material and wherein at least one section does not comprise the fibrous material.

According to a further alternative method, the carrier comprises two or more sections separated by a perforated line and the one or more pieces of fibrous material are placed on the perforated line, and wherein the carrier optionally comprises at least one non-adhesive section.

The perforations of the whole carrier or of the fibrous material may facilitate collecting the fibrous material for further analysis and reduces the risk of contamination. Thereby collecting the material can be more convenient and contamination during removal of the material can be avoided. If the fibrous material is for example separated into two or more segments, e.g. via perforated lines, and only one of the segments is directly in contact with the adhesive part, depending on the design and position of the segments, single segments which are not the one being in direct contact with the adhesive part, can be removed and further analyzed while the other segments stay in place. For example, the segments can be positioned in a row with the segment at one end of the row being attached to the surface via the adhesive part. The other segments are not directly attached to the surface. Single segments can then be removed from the row starting with the one on the other end while the rest remains being attached to the wall via the segment connected with the adhesive part. With this several contamination determinations can be performed by after certain times removing one or more segments without the need to fully substitute the carrier.

Specifically, the adhesive carrier can contain 2, 3, 4, 5, 6, 7, 8, 9, 10 segments containing fibrous material, each segment separated by a perforation line to allow removal of one segment, thereby allowing the other segments to be affixed to the surface for further collecting contaminants.

In a preferred embodiment, the carrier also comprises a coding. The coding may be any kind of ID, label or code which enables traceability or provides any type of information about the carrier, like its composition, charge number, manufacturer, position, time of application etc. The coding may be in the form of a bar code, RFID code, number code, writing, color code etc. It may also be a free spot on the carrier to which the user can apply additional information, e.g. in writing or with stickers. It may also be a label which provides the user with information about the time for which the carrier has already been exposed, e.g. by showing a color change or fading over the time.

Preferably, the coding is used to allow traceability. The coding may be directly applied to the fibrous material or it may be attached to the fibrous material and/or to the adhesive part.

The carrier may also comprise a cover. The cover may be used to cover the fibrous material. This might e.g. be favorable during transportation, for long term sampling or for investigation. Preferably, the cover covers the whole fibrous material. It might be a plastic layer or a plastic sheet that is either permanently fixed to e.g. one side of the carrier and can for coverage be put over the fibrous material and optionally be fixed to one or more of the other sides of the carrier for stable coverage. It can also be a cover sheet that is not permanently fixed to the carrier but can be affixed to it if needed, e.g. by affixing it to the top and the bottom side to ensure enough protection.

The carrier may also be part of a kit which further comprises an instruction leaflet including a protocol for the detection of biological contaminants as described herein and/or reagents for performing the detection,

After collecting the pieces of fibrous material, said material is incubated in a solvent for analysis of contaminants. Any solvent can be used which is suitable to detach the contaminant or its parts or fragments from the fibrous material. Said solvents can be, but are not limited to, buffers, such as buffers containing solvents, surfactants, detergents, or buffers without solvents, surfactants, detergents, Tris/EDTA; chaotropic solvents, organic solvents, ionic liquids.

The term “incubating” refers to the time of contacting the fibrous material with the solvent to detach contaminants from the material. Incubation time may range from 1, 2, 3, 4, 5, or more minutes but can be several days or weeks if the sample is stored for further analysis.

The solvent is then analyzed for the microbes or parts thereof such as proteins, peptides, carbohydrates, lipids, small molecules, cellular organic and inorganic compounds, and nucleic acid molecules, specifically DNA or RNA and any combinations thereof. The microbes and parts thereof can also be further processed for analysis.

The presence of contaminants can be determined by polymerase chain reaction (PCR). PCR methods are well known in the art and are widely used. They include quantitative PCR, semi-quantitative PCR, multiplex PCR, digital PCR, or any combination thereof. In a particularly preferred embodiment, quantitative PCR (q-PCR) is used. An overview of real time PCR quantification methods is given by Schmittgen et al., 2008, Methods. January; 44(1): 31-38.

As an example, once the sample is collected, DNA or RNA is isolated and extracted from the sample. The isolated DNA may be divided into small portions and placed in a reaction vessel, such as, e.g., a PCR tube, with appropriate PCR reagents. Each reaction vessel may also receive a pair of primers, a pair of oligonucleotide probes, an internal control (IC) construct, and a pair of probes for the internal control and target. The primers and probes may be specific for a single species under examination. The PCR reagents, primers, probes, and IC may be provided in a mixture or ready-to-use form, e.g., in a solution or as a freeze-dried mixture. The internal control may also be amplified by the species-specific primer, but it is detected with its own unique probes. With the availability of primer and probe pairs for multiple species, the isolate from a single sample may be tested for the presence of multiple species of interest.

According to an alternative method, next generation sequencing (NGS), enzyme-linked immunosorbent assay (ELISA) or other immunoassays can be performed.

The present method provides a highly efficient and sensitive tool for long-term monitoring of areas. Thereby areas within facilities can be repeatedly or continuously supervised by applying pieces of fibrous material as described herein to selected surfaces and performing the method as described herein.

Further provided is a ready-to-use carrier comprising at least one surface with a sterile, preferably nucleotide free fibrous material. In one embodiment, the carrier is made like a sticker and comprises a second, opposite surface which is partly or fully adhesive. For storage and handling prior to use this carrier is covered by a top and bottom sterile protective layer and/or is within a sterile packaging like a bag. An exemplary schematic picture is given in FIG. 7. Such sticker allows application of the fibrous material without contamination due to handling. Additionally, if provided as a ready to use kit, an instruction leaflet including a protocol for the detection of biological contaminants as described herein is included. The kit may also include reagents for performing the detection. According to a specific example such ready-to-use sticker comprises an upper protective layer with position indicator (1) and a non-adhesive section (2A)(2B), self-adhesive fibrous material (3), an adhesive carrier with a non-adhesive section (5A)(5B) and a perforated line (6), and a lower protective layer with a layer separation guiding line (7). The lower protective layer is removed before application of the sticker to allow sticking the adhesive carrier to the sampling surface. Specifically, upon removal of the upper protective layer monitoring of contamination is commenced. Specifically, the non-adhesive sections (5A) and (5B) can be seized, for example with forceps, for removal of the sticker from the sampling surface. Specifically, the perforated line (6) allows creasing of the adhesive carrier for easier removal of the fibrous material (3).

The following items are particular embodiments described herein.

1. A method for the detection of biological contaminants on a surface, comprising the sequential steps of
i. providing a carrier with one or more pieces of sterile and nucleotide-free adhesive fibrous material and an adhesive part,
ii. affixing said carrier to said surface,
iii. collecting at least one piece of the fibrous material from said surface. The carrier might be collected as a whole or parts or the whole or parts of the fibrous material might be collected,
iv. incubating said at least one piece of the fibrous material in a solvent, and
v. analyzing the solvent for the presence of biological contaminants.

2. The method of item 1, wherein at least 2 pieces of fibrous material are used, specifically at least 3, 4, 5 or 6 pieces of fibrous material are used.

3. The method of item 1 or 2, wherein the fibrous material is comprised on an adhesive support capable of adhering to the surface.

4. The method of item 1 or 2, wherein the fibrous material is comprised on a layer of paper which is adhered to an adhesive support capable of adhering to the surface.

5. The method of any one of items 1 to 4, wherein the carrier comprises at least two sections, optionally separated by a perforated line, wherein at least one section comprises one or more pieces of sterile and nucleotide-free adhesive fibrous material and wherein at least one section does not comprise the fibrous material.

6. The method of any one of items 1 to 4, wherein the carrier comprises at least two sections separated by a perforated line and the one or more pieces of fibrous material are situated on the perforated line, and wherein the carrier optionally comprises at least one non-adhesive section.

7. The method of any one of items 1 to 6, wherein the biological contaminants are bacteria, specifically Listeria monocytogenes or E. coli, yeast or viruses.

8. The method of any one of items 1 to 7, wherein the solvent is selected from the group consisting of buffers, specifically selected from the group of buffers with solvents, surfactants, detergents, buffers without solvents, surfactants, detergents, Tris/EDTA; chaotropic solvents, organic solvents, ionic liquids.

9. The method of any one of items 1 to 8, wherein the solvent is analyzed for parts of a biological contaminant selected from the group consisting of proteins, peptides and nucleic acid molecules, specifically DNA or RNA.

10. The method of any one of items 1 to 9, wherein the solvent is analyzed for a biological contaminant or parts of a biological contaminant using PCR, qPCR, next generation sequencing (NGS), enzyme-linked immunosorbent assay (ELISA) or other immunoassays.

11. The method of item 9, wherein the biological contaminant is L. monocytogenes and the solvent is analyzed for the presence of the L. monocytogenes gene prfA.

12. The method of item 7, wherein the biological contaminant is E. coli and the solvent is analyzed for the presence of the E. coli gene sfmD.

13. The method of any one of items 1 to 12, wherein the fibrous material is affixed to the surface for at least 1 hour, 6 hours, 8 hours or 12 hours, preferably at least 24 hours.

14. The method of any one of items 1 to 12, wherein the fibrous material is affixed to the surface for at least a week, preferably at least 2 weeks.

15. The method of any one of items 1 to 14, wherein the fibrous material, the adhesive carrier and the layer of paper are sterilized using a physical or chemical sterilization method, specifically selected from the group consisting of UV radiation, gamma radiation, electron beam radiation, X-ray radiation, radiation with subatomic particles, plasma, dry heat, autoclaving, ozone, hydrogen peroxide, peracetic acid, nitrogen dioxide, ethylene oxide, hypochlorite and DNase.

16. The method of any one of items 1 to 15, wherein the fibrous material is inorganic or organic fibrous material, specifically selected from the group consisting of activated carbon, microporous ceramic, porous metal, aluminum oxide, glass fiber, paper, cellulose, cellulose esters, cellulose ethers, cellulose acetate, viscose, cellophane, alginate, nylon membranes, polyester (PETE), polypropylene, polytetrafluoroethylene (PTFE), polyvinylidene fluoride, polyvinylidene difluoride (PVDF), polycarbonate (PCTE), polyether ether ketone (PEEK), polyacrylonitrile (PAN), polyaramide (KEVLAR), and polyethersulfone (PES).

17. The method of any one of items 1 to 16, wherein the adhesive support is selected from the group consisting of adhesive tape, specifically selected from the group consisting of polyethylene film, polypropylene film, polyester film, polyvinyl chloride (PVC), Cellulose film, plastic paraffin film, and metal foil.

18. The method of any one of items 1 to 17, wherein the one or more pieces of fibrous material comprise a surface area of at least 10 mm², preferably 50 to 300 mm², more preferably 50 to 100 mm².

19. The method of any one of items 1 to 18, wherein the one or more pieces of fibrous material, the layer of paper and/or the adhesive carrier are supplemented with a bacteriostatic and/or bacteriocide composition.

20. Use of the method of any one of items 1 to 19 for long-term monitoring of biological contaminants.

21. Use of the method of any one of items 1 to 20 for monitoring, specifically long-term monitoring, of biological contaminants in the food industry or in the medical or pharmaceutical sector.

22. A kit of parts comprising at least the following parts:
i. at least one carrier, preferably in form of a sticker, comprising a sterile carrier comprising a first and second surface, wherein said first surface is adhesive and said second surface comprises at least one piece of sterile and nucleotide-free adhesive fibrous material and wherein said sticker is covered by a top and bottom sterile protective layer, and
ii. an instruction leaflet including a protocol for the detection of biological contaminants according to any one of items 1 to 21 and/or reagents for performing the detection.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

Regular sampling is mandatory for proper monitoring of microbes in sensitive environments. This procedure is commonly performed using swabs. Besides a rather small recovery, detection results obtained only represent a momentary snapshot and subsequent disinfection after sampling is advised, which makes the sampling process laborious. Therefore, in this study, an alternative surface suitable for trapping bacteria was examined. Attached to the surface of interest as a sticker, it can remain in place for long time periods while collecting contaminants. In this manner the appropriateness of text marking stickers was investigated, comprising plain paper surfaces, as an alternative sampling system.

### Example 1: DNA Recovery from Stickers is Sufficient and Constant Over Time

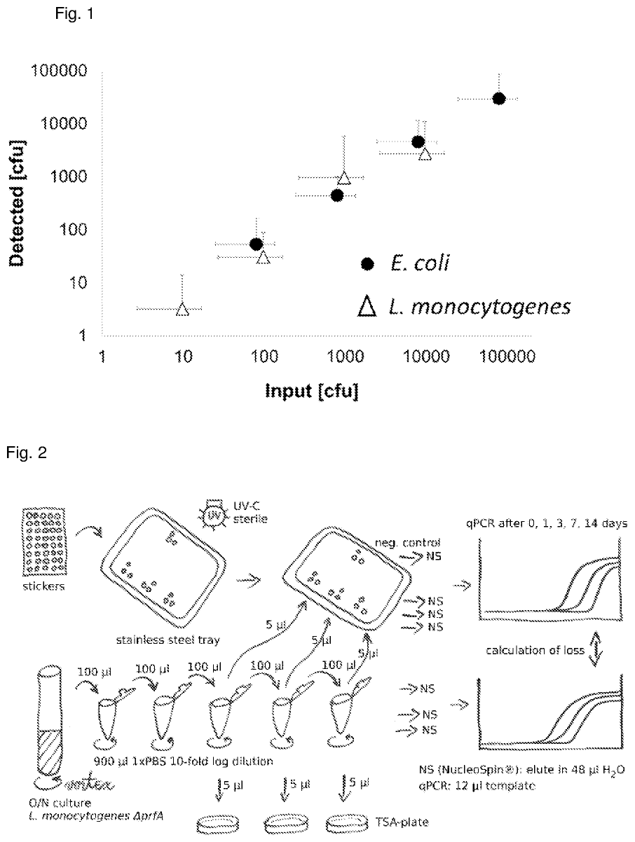

The suitability of text marking stickers as sampling alternatives for swabs was initially assessed quantitatively and qualitatively using both molecular and microbiological methods. L. monocytogenes (AprfA) and E. coli were applied to stickers in concentrations ranging over four logarithmic units. After 24 h the stickers were either quantitatively analyzed using plate count methods or qualitatively with enrichment in TSB or half Fraser medium for microbiological analysis (Table 1). The enrichment method resulted in random detection of E. coli at any concentration while L. monocytogenes was detected from 100 cfu. Poor results were obtained using the plate count method where almost no growth was achieved at any tested concentrations. In contrast, qPCR provided quantitative results for both bacteria at all concentrations, although recovery of L. monocytogenes was lower than for E. coli (FIG. 1).

FIG. 1 displays the quantification of L. monocytogenes and E. coli from artificially contaminated stickers over a broad dynamic range. DNA from stickers artificially contaminated with four 10-fold logarithmic dilutions (starting at 80 cfu for E. coli and 10 cfu for L. monocytogenes) was extracted and quantified using qPCR (y axis). Control DNA (input, applied on stickers) was extracted and analyzed simultaneously as reference (x axis). Symbols and error bars denote standardized mean differences and standard deviation, respectively (n=3 independent experiments with three repetitions each). For the sake of clarity only positive y-error bars of are displayed (negative y-error bar values are identical to the positive values).

Thus, further experiments focused on qPCR analysis as the detection method of choice.

TABLE 1
Growth of L. monocytogenes ΔprfA
and E. coli after drying on stickers.

| | | | |
|---|---|---|---|
| | L. | L. | |
| | monocytogenes | monocytogenes | |
| Input | ΔprfA | ΔpfrA | E. coli |
| (cfu) | in TSB | Half fraser | TSB |

Positive findings out of 9

| | | | |
|---|---|---|---|
| 10 | 1/9 | 0/9 | 1/9 |
| 10² | 8/9 | 5/9 | 1/9 |
| 10³ | 9/9 | 8/9 | 1/9 |
| 10⁴ | 9/9 | 9/9 | 1/9 |

Numbers of positive findings (growth) after stickers incubated in TSB (L. monocytogenes, E. coli) or half Fraser (L. monocytogenes) of three independent experiments performed in triplicate.

To determine stability over time, five sets comprising six sterile stickers were contaminated artificially with different counts of L. monocytogenes (AprfA). Two stickers each were contaminated with 5 cfu, 50 cfu or 500 cfu, respectively, and were analyzed for up to 14 days. For comparative purposes, the same inoculum was plated on TSA-plates or DNA was extracted directly after dilution FIG. 2 shows a schematic representation of the artificially contaminated sticker setup. UV-treated stickers were artificially contaminated by the addition of diluted bacteria suspensions at desired concentrations. After respective incubation times, DNA from stickers and controls was extracted and analyzed using qPCR. In parallel, cells were plated on TSA plates as controls.

Recovery from the stickers was rather variable, at around 30%, but did not distinctly decrease after 14 days demonstrating the possibility of sampling over a prolonged time period.

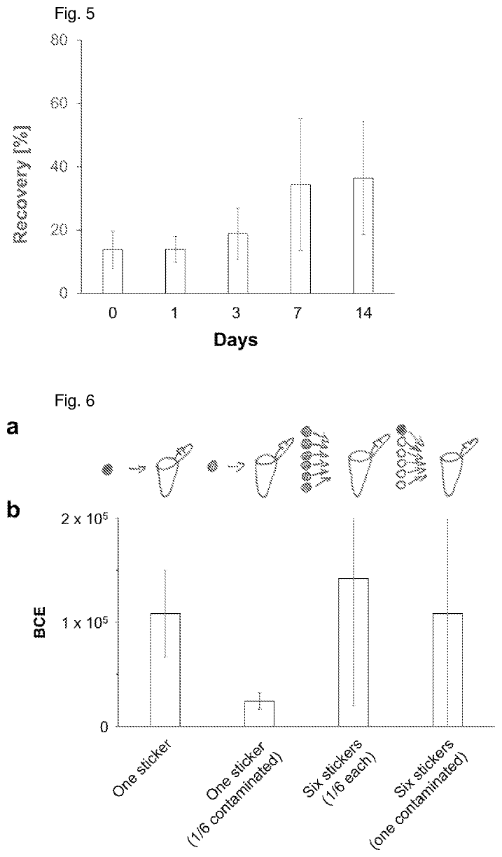

In FIG. 5. stability of recovery over time is shown. Stickers were artificially contaminated with L. monocytogenes 0.6prfA and DNA extracted and analyzed with qPCR after 0, 1, 3, 7 and 14 days. Bars and errors bars represent grand means of recovery (outcome (qPCR)/input (qPCR)) and standard errors of four (days 0, 3) or three (1, 7, 14 days) independent experiments, including two different bacterial concentrations in duplicate.

The results are similar to those obtained from recovery from sponge-sticks in a previous study (Witte A K, et al., 2018).

### Example 2: Cleansing and Disinfection have Minor Impacts on Bacterial Detection with Stickers

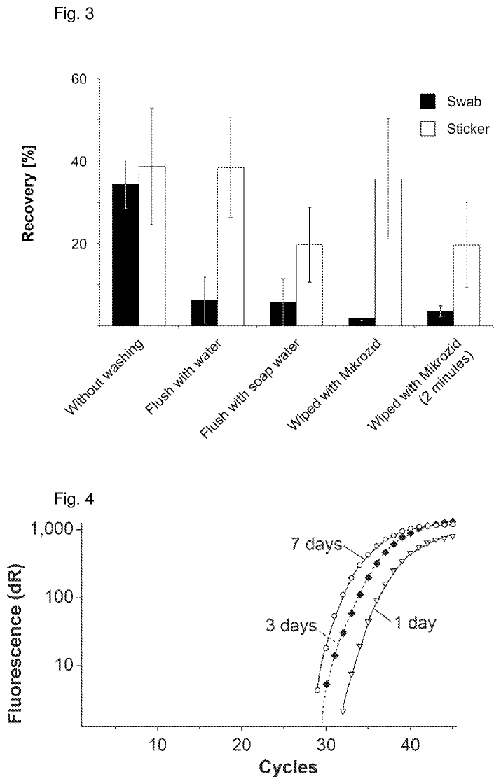

Surfaces in food processing plants are anticipated to be cleansed regularly. Therefore, to test whether the paper stickers convey advantages or disadvantages compared with conventional sampling, artificially contaminated (L. monocytogenes ΔprfA) stickers were treated with water, soap-water and a disinfection agent to simulate routine cleansing practices. As a control, ceramic tiles were artificially contaminated, treated the same way as the stickers and sampled using sponge-stick swabs. The results summarized in FIG. 3 reveal that after cleansing and/or disinfection, distinctly more bacteria could be detected using stickers.

FIG. 3 shows the recovery after cleansing and disinfection. Surfaces or stickers applied to surfaces were artificially contaminated with 10³ to 10⁴ cfu of L. monocytogenes ΔprfA. After drying, surfaces were washed, subsequently sampled and DNA extracted and analyzed with qPCR. Bars represent the grand mean of recovery (outcome (qPCR)/input (qPCR)) with the standard error of five independent experiments performed in duplicate.

### Example 3: Pooling of Up to Six Stickers

The surface of one sticker measured only 50 mm², which is much smaller than commonly suggested for swabbing. In order to optimize sampling density versus effort (labour power and materials), it was decided to process more than one sticker per DNA extraction sample rather than using larger stickers. Although the number of stickers to be processed at once is restricted by the volume of pre-lysis buffer used in the first step of the NucleoSpin® kit, it was found that six stickers per sample proved to be a good compromise when retaining the original protocol. To test whether pooling six stickers leads to loss of information due to possible dilution by empty stickers or increased quantities of insoluble material, two pools were tested against reference samples (FIG. 6a).

FIG. 6 shows the pooling of stickers. a. Schematic representation of the pooling approach demonstrates the different samples: one single contaminated sticker, a single sticker with 1/6 contamination level, a pool containing six contaminated stickers and a pool containing one contaminated and five empty stickers. b. Results show that pooling of six stickers does not lead to a great loss of information. BCE (bacterial cell equivalents) were determined using qPCR. Bars represent the standardized mean difference with the standard deviation of four independent experiments.

Both pools contained the same bacteria count. A single sticker carrying the same or 1/6 of the contamination level as both pools served as control. As demonstrated in FIG. 6b, pooling appeared to be adequate whereby similar results were obtained independent of the number of (empty or contaminated) stickers. Despite relatively high variation, only the total amount of DNA in the sample appears to be relevant. Thus, this pooling approach might help to reduce sample numbers. Statistically a higher number of applied stickers increases the chance of detecting minor contaminants.

### Example 4: Sampling On-Site and Proof of Concept for Successful Detection of Bacterial Contamination Using Stickers

After investigating the suitability of the new method with artificial contamination experiments, the stickers were utilized in a proof of concept experiment to establish whether bacteria can be captured with this system. For this purpose, stickers were applied at several locations that underwent frequent hand contact, such as door handles or light switches of toilets, for one to seven days. In the first setup, for detecting L. monocytogenes, sampling using sponge-sticks was performed in parallel. In the second setup, qPCR for E. coli was additionally performed to supplement the occurrence of positive results and to monitor another species. Swabbing was omitted in this setup, but three time periods were included. Since it was demonstrated that the prfA assay can detect and quantify even down to one single molecule (Rossmanith P, and Wagner M., J Food Prot. 2011; 74(9):1404-1412.), each positive signal in qPCR was rated as a positive result.

Results summarized in Tables 2 and 3 show that stickers detected both bacterial species repeatedly from several locations suggesting suitability as an appropriate on-site sampling/detection system. Further, in the first setup the stickers detected similar or even higher occurrences of L. monocytogenes compared with the conventional swab system (Table 2). Finally, an analysis of stickers that were applied for periods of one to seven days indicate their suitability for sampling and detection, essentially independently of the date of contamination (Table 3).

TABLE 2
Detection of L. monocytogenes on-site using stickers and swabs

| Location | | | | | | | | | |
|---|---|---|---|---|---|---|---|---|---|
| | | | | | site | site | site | site | site |
| site 1 | site 2 | site 3 | site 4 | site 5 | 6 | 7 | 8 | 9 | 10 |

Positive findings out of 7

| | | | | | | | | | | |
|---|---|---|---|---|---|---|---|---|---|---|
| Sticker | 2/7 | 2/7 | 3/7 | 7/7 | 2/7 | 5/7 | 3/7 | 4/7 | 0/7 | 2/7 |
| Swab | 1/7 | 0/7 | 2/7 | 1/7 | 1/7 | 1/7 | 0/7 | 1/7 | 0/7 | 2/7 |

Number of L. monocytogenes positive findings in qPCR of seven independent trials (Setup 1).

TABLE 3
Detection of L. monocytogenes and E. coli on-site with stickers

| E. coli | | | L. monocytogenes | | |
|---|---|---|---|---|---|
| 1^{st} pass | 2^{nd} pass | 3^{rd} pass | 1^{st} pass | 2^{nd} pass | 3^{rd} pass |

Positive findings out of 7

| | | | | | | |
|---|---|---|---|---|---|---|
| 1 day | 4/5 | 2/5 | 0/5 | 1/5 | 1/5 | 0/5 |
| 3 days | 4/4* | 2/5 | 0/5 | 1/4* | 0/5 | 0/5 |
| 7 days | 3/4* | 1/5 | 1/5 | 0/4* | 1/5 | 0/5 |

Number of positive findings in qPCR on five door handles tested three times, including three time periods (Setup 2).
*one sticker was lost

### Example 5: Accumulation of Free DNA on Frequently Used Door Handles

Besides the monitoring of microbes in our proof of concept study, the prfA IAC assay was examined in parallel because the lyophilised internal amplification control was accidently distributed in one room more than 10 years ago. Startlingly, this synthetic oligonucleotide of 100 base pairs could still be detected on the door handle of this room, and even accumulated over time on the stickers, demonstrating the stability of DNA and the ability of the new sticker system to detect it effectively. FIG. 4 shows the accumulation of synthetic IAC on stickers over time qPCR (IAC assay) of DNA extracted from stickers applied to a door handle demonstrates an accumulation over time of synthetic DNA on stickers that was distributed in this room. Results are representative of three independent experiments.

### Discussion

Although there was variation in recovery from artificially contaminated stickers, the stickers provided results similar to those previously obtained with swabs. Preservation of DNA on the sticker also showed the method to be very reliable over time. The presented detection is qPCR-based. No inhibitory factors impairing DNA-extraction or qPCR were encountered. However, as positive findings are a statement for the presence of DNA, this does not inevitably originate from living cells. Therefore the method thus detects living, dead and viable but non-culturable cells (VBNC). While detection of non-growing cells in the past was often discussed as a disadvantage, increasing interest and awareness of VBNCs today highlights that non-growing cells are also a potential threat (Silva S, et al. 2008). Further, detection of non-growing cells advantageously attests to badly cleansed areas as most chemical disinfectants alone cannot remove DNA.

As shown in the proof of concept experiment, synthetic DNA is effectively captured. Thus, used stickers can also be used to detect “flying” DNA that can also produce severe problems. Contamination of this nature might occur more often than anticipated in establishments and has also been demonstrated with peptides. Although contamination with artificial DNA is unlikely to occur in the food industry, where it is rarely used, there is still a risk of contamination with PCR products. Guidelines for laboratory practice must be followed strictly to minimize risks, e. g. neither to open nor autoclave PCR-tubes prior to disposal. All essential controls must be included in the monitoring setup.

Since regular cleansing and disinfection of surfaces is obligatory in food processing environments, the applicability of stickers after washing was tested and compared to swabbing results from surfaces. Swabs showed similar results prior to cleansing, but the yield from the stickers tended to be higher. This might be secondary to the adherent nature of the stickers themselves that have a good affinity for attaching bacteria. Nevertheless, it must be acknowledged that despite advantages, the sticker itself might have the potential to distribute contaminants, even while studies have shown higher microbial transfer rates from nonporous surfaces (Anonymus. Geneva, Switzerland.; 2004) and no outbreaks from paper as the source of contamination are documented. Reproducible regrowth of L. monocytogenes and E. coli attracted to stickers was observed only at high concentrations (Table 1). However, to circumvent this possible hazard in future, stickers supplemented with bacteriostatic components might be beneficial. Despite offering promising data, further tests on-site are necessary to complete datasets. Initial experiments on-site did show inconsistencies in sticker compound stability. In some cases, the stickers became dog-eared and detached spontaneously from the adhesive tape. A slight improvement was obtained by prior preparation of the compound on a surface of similar geometry to the door handles to which they were subsequently applied. Yet, as many stickers remained fast without any inconsistencies, this problem is not insurmountable.

Despite the small surface area of stickers, measuring only 0.5 cm², even more positive samples were obtained from them compared with swabs. The swabbed area in comparison was at least ten-times greater, demonstrating the capabilities of the sticker system.

### Conclusion

A newly developed sticker system to sample surfaces for microbial contamination, and suitable for molecular detection methods, has demonstrated several advantages over the sponge stick swabbing system, despite comparable losses and variation in recoveries. A major advantage of stickers is in handling: they are easy to distribute and to collect, and no further processing steps, such as centrifugation, are necessary for subsequent DNA-extraction. Results also indicate that cleansing and disinfection only slightly impair results obtained from the stickers, suggesting that prolonged interval sampling should be possible. Additionally, it is not necessary to disinfect monitored surfaces after usage as should be the case when using sponge stick swabs. The presented detection system appears to be a promising alternative for effective sampling of bacterial contaminations.

Materials and Methods

Materials and Methods Used Throughout the Examples:

Bacterial Strains and Growth Conditions

Listeria monocytogenes EGDe and ΔprfA (part of the collection at the Institute of Milk Hygiene, Milk Technology and Food Science, Department for Farm Animal and Public Health in Veterinary Medicine, Vetmeduni Vienna, Austria) and Escherichia coli TOP10F′(Invitrogen, Carlsbad, Calif., USA) were grown overnight in tryptone soya broth with 0.6% (w/v) yeast (TSB-Y; Oxoid, Hampshire, UK) at 37° C.; optical density was measured at 610 nm with a HP 8452 spectrophotometer (Hewlett-Packard, Waldbronn, Germany; 0.6 OD₆₁₀ approximates 10⁸ cfu/ml).

DNA Standard

As DNA standard for qPCR quantification, one milliliter of an L. monocytogenes (strains EGDe or ΔprfA) or E. coli overnight culture was used for DNA isolation with the NucleoSpin® tissue kit (MACHEREY-NAGEL GmbH & Co. KG, Düren, Germany) following protocol instructions for Gram-positive bacteria. The DNA was eluted twice with 50 μl ddH₂O (70° C.). The DNA concentration was measured using the Qubit ds Broad Range Kit (Fisher Scientific, Vienna, Austria). The copy number of the single copy gene (EGDe, E. coli) or single-integrated internal amplification control was calculated using the DNA molecular weight of L. monocytogenes (1 ng of DNA equals 3.1×10⁵ copies of the genome) or E. coli (1 ng of DNA equals 1.8×10⁵ copies of the genome).

qPCR

The prfA qPCR assay for detecting L. monocytogenes was modified after Rossmanith et al. (Res Microbiol. 2006; 157(8):763-771): One qPCR reaction of 25 μl final volume contained 1× reaction buffer (Fisher Scientific, Vienna, Austria), 3.5 mM MgCl₂, 0.5 μM of each primer (Table 4), 0.25 μM of each probe (Table 4), 200 μM each dATP, dTTP, dGPT, and dCTP, 1.5 U of Platinum Taq (Fisher Scientific, Vienna, Austria) and 12 μl of template DNA.

The sfmD qPCR assay for detection of E. coli was modified after Kaclíkovã et al. (Lett Appl Microbiol. 2005; 41(2):132-135): One qPCR reaction of 25 μl final volume contained 1× reaction buffer, 3.5 mM MgCl₂, 0.3 μM of each primer (Table 4), 0.2 μM of probe (Table 4), 200 μM each dATP, dTTP, dGPT, and dCTP, 1 U of Platinum Taq (Fisher Scientific, Vienna, Austria) and 12 μl of template DNA.

The qPCR was performed as previously published in an Mx3000p real-time PCR thermocycler (Stratagene, La Jolla, Calif., USA) using the thermal programs listed in Table 4 and the analysis was performed with MxPro software (adaptive baseline settings).

TABLE 4
Primers, probes and thermal program of qPCR assays

| | | | Length of | |
|---|---|---|---|---|
| | | Sequence (5′-3′) | product | PCR |
| Assay | | Primers and probes | (bp) | program |
| prfA assay | Lip1 | 5′-GAT ACA GAA ACA TCG GTT | 274 | 94° C., |
| (L. mono- | | GGC-3′ | | 2 min |
| cytogenes) | | (SEQ ID NO: 1) | | 45 × |
| | Lip2 | 5′-GTG TAA TCT TGA TGC CAT | | [94° C., |
| | | CAG G-3′ | | 15 s; |
| | | (SEQ ID NO: 2) | | 64° C., |
| | LIP Probe2 | 5′-FAM-CAG GAT TAA AAG TTG | | 60 s] |
| | | ACC GCA-BHQ1-3′ | | |
| | | (SEQ ID NO: 3) | | |
| IAC assay | Lip1 | 5′-GAT ACA GAA ACA TCG GTT | 100 | 94° C., |
| (L. mono- | | GGC-3′ | | 2 min |
| cytogenes | | (SEQ ID NO: 4) | | 45 × |
| ΔprfA or | Lip2 | 5′-GTG TAA TCT TGA TGC CAT | | [94° C., |
| synthetic | | CAG G-3′ | | 15 s; |
| IAC) | | (SEQ ID NO: 5) | | 64° C., |
| | p-lucLm 5 | 5′-HEX-TTC GAA ATG TCC GTT | | 60 s] |
| | | CGG TTG GC-BHQ1-3′ | | |
| | | (SEQ ID NO: 6) | | |
| sfmD assay | Ert2F | 5′-ACT GGA ATA CTT CGG ATT | 106 | 94° C., |
| (E. coli) | | CAG ATA CGT-3′ | | 2 min |
| | | (SEQ ID NO: 7) | | 50 × |
| | Ert2R | 5′-ATC CCT ACA GAT TCA TTC | | [94° C., |
| | | CAC GAA A-3′ | | 15 s; |
| | | (SEQ ID NO: 8) | | 60° C., |
| | Ert2P | 5′-FAM-CAG CAG CTG GGT TGG | | 60 s] |
| | | CAT CAG TTA TTC G-BHQ1-3′ | | |
| | | (SEQ ID NO: 9) | | |

All primers and probes were obtained from Eurofins (Ebersberg, Germany).

Stickers

Commercially available text marking stickers (Markierungspunkte Ø 8 mm, permanent, No. 3013 yellow, 3175 white, 3179 green, AVERY™ of CCL Industries Inc., Toronto, Canada) were applied to a strip of adhesive tape (Tesafilm® transparent, 15 mm No. 57370-02, tesa SE, Norderstedt, Germany) using sterile tweezers followed by sterilization with UV-C radiation for 15 min (Sylvania G30W T8, 10 cm distance, Feilo Sylvania, Erlangen, Germany). The sticker compound was then attached to the surface of interest.

Artificial Contamination of Stickers

For artificial contamination of stickers (FIG. 2), bacteria were washed and log-diluted in 1×PBS (phosphate-buffered saline). A 5 μl droplet of the respective bacterial suspension was applied to each sticker to achieve approximately 10, 100, 1000 or 10,000 colony forming units (cfu) per sample. Bacterial suspensions were dried for at least one hour or until any visible moisture had evaporated or they were kept at room temperature for 1, 3, 7 or 14 days. After the respective storage times, stickers were transferred into a 1.5 ml Eppendorf tube using sterile tweezers for subsequent DNA extraction. As references, an equi-volume inoculum was transferred directly to DNA extraction and to TSA-Y plates to obtain reference values. In parallel, artificially contaminated stickers were incubated for one hour in 500 μl 1×PBS, vortexed and the entire supernatant plated to TSA-Y. Alternatively, stickers were transferred to half Fraser broth (BIOKAR Diagnostics, Beauvais, France) or TSB medium and bacterial growth assessed after 24 h at 30° C. or respectively 37° C.

Experiments were performed at room temperature (22° C. to 25° C.) and relative humidity levels were between 40 and 60%.

DNA Recovery and Isolation from Stickers

Stickers detached with sterile tweezers and transferred into 1.5 ml Eppendorf tubes were used directly for DNA extraction with the NucleoSpin® Tissue DNA extraction kit (MACHEREY-NAGEL GmbH & Co. KG, Düren, Germany) by adding the pre-lysis buffer on top of the stickers. The original protocol for Gram-positive bacteria was followed with the modification of DNA elution with twice 24 μl ddH₂O (70° C.) in order to reduce the volume, yielding a 48 μl elution volume instead of 100 μl.

Sampling with Sponge-Sticks

The performance of stickers was compared with sponge stick swabbing (Sponge-Stick with Buffered Peptone Water Broth, 3M™, St. Paul, Minn., USA). After surface sampling, sponges were soaked with 10 ml 1×PBS and stomached for 2 min. The liquid was centrifuged for 5 min at 8,000 g, and the obtained pellet subsequently used for DNA-extraction with the NucleoSpin® kit (elution with twice 48 μl H₂O, 70° C.).

Cleansing and Disinfection

Soap water was prepared by diluting EXACT AC (E. Mayr, Vösendorf, Austria) in water to concentrations commonly using for cleansing surfaces. For disinfection of surfaces, Mikrozid® AF liquid (Schülke & Mayr, Norderstedt, Germany) was applied by wiping. Two minutes exposure times were also tested for comparison.

1. A method for the detection of biological contaminants on a surface, comprising the sequential steps of
i. providing a carrier comprising one or more pieces of sterile fibrous material and an adhesive part for affixing said carrier to a surface,
ii. affixing said carrier to said surface,
iii. collecting at least one piece of the fibrous material from said surface,
iv. incubating said at least one piece of the fibrous material in a solvent, and
v. analyzing the solvent for the presence of biological contaminants.

2. The method of claim 1, wherein at least 2 pieces of fibrous material are used, specifically at least 3, 4, 5 or 6 pieces of fibrous material are used.

3. The method of claim 1, wherein the adhesive part is an adhesive applied to at least part of one side of the sterile fibrous material, a layer of paper, plastic or metal with an adhesive, or a plastic or metal holding that can be fixed to a surface.

4. The method of claim 1, wherein the carrier comprises at least two sections, optionally separated by a perforated line.

5. The method of claim 1, wherein the biological contaminants are bacteria, specifically Listeria monocytogenes or E. coli, fungi or viruses, and the solvent is analyzed for parts of a biological contaminant selected from the group consisting of proteins, peptides and nucleic acid molecules, specifically DNA or RNA.

6. The method of claim 1, wherein the solvent is selected from the group consisting of buffers, specifically selected from the group of buffers with solvents, surfactants, detergents, buffers without solvents, surfactants, detergents, Tris/EDTA; chaotropic solvents, organic solvents, ionic liquids.

7. The method of claim 1, wherein the solvent is analyzed for a biological contaminant or parts of a biological contaminant using PCR, qPCR, next generation sequencing (NGS), enzyme-linked immunosorbent assay (ELISA) or other immunoassays.

8. The method of claim 1, wherein the biological contaminant is L. monocytogenes and the solvent is analyzed for the presence of the L. monocytogenes gene prfA and/or the biological contaminant is E. coli and the solvent is analyzed for the presence of the E. coli gene sfmD.

9. The method of claim 1, wherein the fibrous material is affixed to the surface for a time between 1 hour and 2 weeks.

10. The method of claim 1, wherein the carrier is sterilized using a physical or chemical sterilization method, specifically selected from the group consisting of UV radiation, gamma radiation, electron beam radiation, X-ray radiation, radiation with subatomic particles, plasma, dry heat, autoclaving, ozone, hydrogen peroxide, peracetic acid, nitrogen dioxide, ethylene oxide, hypochlorite and DNase.

11. The method of claim 1, wherein the fibrous material is inorganic or organic fibrous material, specifically selected from the group consisting of activated carbon, microporous ceramic, porous metal, aluminumoxide, glass fiber, paper, cellulose, cellulose esters, cellulose ethers, cellulose acetate, viscose, cellophane, alginate, nylon membranes, polyester (PETE), polypropylene, polytetrafluoroethylene (PTFE), polyvinylidene fluoride, polyvinylidene difluoride (PVDF), polycarbonate (PCTE), polyether ether ketone (PEEK), polyacrylonitrile (PAN), polyaramide (KEVLAR), and polyethersulfone (PES), and wherein the adhesive part is selected from the group consisting of adhesive tape, specifically selected from the group consisting of polyethylene film, polypropylene film, polyester film, polyvinyl chloride (PVC), Cellulose film, plastic paraffin film, and metal foil.

12. The method of claim 1, wherein the one or more pieces of fibrous material comprise a surface area of at least 10 mm², preferably 50 to 300 mm², more preferably 50 to 100 mm².

13. A method of for monitoring, specifically long-term monitoring, of biological contaminants, comprising performing the method according to claim 1 periodically through the time of monitoring.

14. A carrier comprising one or more pieces of fibrous material and an adhesive part for affixing said carrier to a surface whereby the carrier is sterile and comprises a coding.

15. The carrier according to claim 14 whereby the carrier is supplemented with a bacteriostatic and/or bacteriocide composition.

16. The method according to claim 12, wherein the long-term monitoring is from 48 hours up to 4 weeks.
